(19) United States
(12) Patent Application Publication
(10) Pub. No.: US 2024/0201202 A1
(43) Pub. Date: Jun. 20, 2024

(54) METHODS FOR DETECTING IMMUNOGLOBULIN G, SUBCLASS 4 (IgG4) IN A BIOLOGICAL SAMPLE

(71) Applicant: Abbott Laboratories, Abbott Park, IL (US)

(72) Inventors: Nicolette Jeanblanc, Abbott Park, IL (US); Susan Brophy, Abbott Park, IL (US); Bryan TIEMAN, Abbott Park, IL (US); Himali SONI, Abbott Park, IL (US); Salman ALI, Abbott Park, IL (US)

(21) Appl. No.: 18/586,721

(22) Filed: Feb. 26, 2024

Related U.S. Application Data
(63) Continuation of application No. PCT/US22/41451, filed on Aug. 25, 2022.
(60) Provisional application No. 63/237,725, filed on Aug. 27, 2021.

(51) Int. Cl. G01N 33/68; G01N 21/59; G01N 33/543

(52) U.S. Cl. CPC G01N 33/6854; G01N 21/59; G01N 33/54313

(57) ABSTRACT

Disclosed herein are methods, kits, and systems for detecting or determining an amount, quantity, concentration and/or level of immunoglobulin G, subclass 4 (LgG4) in a biological sample from a subject. Particularly, the methods, kits and systems are directed to detection of IgG4 using an anti-IgG4 antibody that does not cross-react with other IgG subclasses.

### RELATED APPLICATION INFORMATION

This application is a continuation of International Application No. PCT/US2022/041451, filed Aug. 25, 2022, which claims the benefit of U.S. Provisional Application No. 63/237,725, filed Aug. 27, 2021, the contents of which are herein incorporated by reference in their entirety.

### TECHNICAL FIELD

The present disclosure relates methods, kits, and systems for detecting or determining an amount, quantity, concentration and/or level of immunoglobulin G, subclass 4 (IgG4) in a biological sample from a subject.

### BACKGROUND

The main immunoglobulin (Ig) in human blood is IgG. This is the second most abundant circulating protein and contains long-term protective antibodies against many infectious agents. IgG is a combination of four slightly different types of IgG called IgG subclasses: IgG1, IgG2, IgG3 and IgG4. The IgG subclass proteins consisting of IgG1, IgG2, IgG3 and IgG4 are more than 95% homologous in amino acid sequence.

While all the IgG subclasses contain antibodies to components of many disease-causing bacteria and viruses, each subclass serves a slightly different function in protecting the body against infection. For example, IgG1 and IgG3 subclasses are rich in antibodies against proteins such as the toxins produced by the diphtheria and tetanus bacteria, as well as antibodies against viral proteins. In contrast, IgG2 antibodies are predominantly against the polysaccharide (complex sugar) coating (capsule) of certain disease-producing bacteria, such as, Streptococcus pneumoniae and Hemophilus influenzae.

The IgG in the bloodstream is 60-70% IgG1, 20-30% IgG2, 5-8% IgG3 and 1-3% IgG4. The amount of the different IgG subclasses present in the bloodstream varies with age. For example, IgG1 and IgG3 reach normal adult levels by 5-7 years of age while IgG2 and IgG4 levels rise more slowly, reaching adult levels at about 10 years of age. When one or more of these subclasses is persistently low but total IgG is normal, a subclass deficiency is present.

IgG subclass deficiencies may be associated with poor or partial responses to infections and inflammatory diseases and often result in an increased in recurrence of infections or inflammation. Thus, accurate assessment each IgG subclass, as well as total IgG, allows full characterization of a patients immunoglobulin status, and may provide much needed insight into patient experience an increase in recurrent infections or inflammation.

### SUMMARY

In one embodiment, the present disclosure relates to a method of detecting immunoglobulin G, subclass 4 (IgG4) in a biological sample. The method comprises performing an assay on the biological sample obtained from a subject, wherein the assay includes adding an anti-IgG4 antibody capture reagent to the biological sample, wherein the anti-IgG4 antibody capture reagent comprises an anti-IgG4 antibody immobilized on a solid support, wherein the anti-IgG4 antibody does not cross-react with other IgG subclasses.

In one aspect of the above method, the assay further comprises incubating the anti-IgG4 antibody capture reagent with the biological sample for a period of time.

In another aspect of the above method, the assay further comprises measuring turbidity. In still a further aspect, the assay comprises comparing at least one turbidity measurement to a standard and quantifying an amount of the IgG4 in the biological sample. In some aspects, the turbidity can be measured by loss of intensity or increase in absorbance of transmitted light through the biological sample at a wavelength of about 570 nm.

In yet a further aspect of the above method, the anti-IgG4 antibody is a monospecific antibody. In some aspects, the monospecific antibody is directed to a linear epitope. In still further aspects, the monospecific antibody is directed to a conformational epitope.

In yet further aspects of the above method, the anti-IgG4 antibody is a monoclonal antibody.

In still yet further aspects of the above method, the anti-IgG4 antibody is selected from the group consisting of: 3H293, 0.B.27, 5C7, IGHG4/1345 and IGHG4/2042A.

In still further aspects of the above method, the solid support is a microparticle. In some aspects, the microparticle comprises latex.

In yet further aspects of the above method, the assay is performed in about 1 minute, in about 5 minutes, in about 10 minutes, in about 15 minutes or in about 20 minutes.

In yet still further aspects of the above method, the biological sample is whole blood, serum, or plasma.

In yet still further aspects of the above method, the assay is an immunoassay or a clinical chemistry assay.

In yet still further aspects of the above method, the method is performed using single molecule detection, lateral flow, or a point-of care method.

In yet still further aspects of the above method, the method is adapted for use in an automated system or a semi-automated system.

In another embodiment, the present disclosure relates to an improvement of a method of detecting immunoglobulin G, subclass 4 (IgG4) in a biological sample, wherein the method comprises performing an assay on the biological sample obtained from a subject, wherein the assay includes adding an anti-IgG4 antibody capture reagent to the biological sample, wherein the anti-IgG4 antibody capture reagent comprises an anti-IgG4 antibody immobilized on a solid support, wherein the improvement comprises using an anti-IgG4 antibody that does not cross-react with other IgG subclasses.

In one aspect of the above improvement, the assay further comprises incubating the anti-IgG4 antibody capture reagent with the biological sample for a period of time.

In another aspect of the above improvement, the assay further comprises measuring turbidity. In still a further aspect, the assay comprises comparing at least one turbidity measurement to a standard and quantifying an amount of the IgG4 in the biological sample. In some aspects, the turbidity can be measured by loss of intensity or increase in absorbance of transmitted light through the biological sample at a wavelength of about 570 nm.

In yet a further aspect of the above improvement, the anti-IgG4 antibody is a monospecific antibody. In some aspects, the monospecific antibody is directed to a linear epitope. In still further aspects, the monospecific antibody is directed to a conformational epitope.

In yet further aspects of the above improvement, the anti-IgG4 antibody is a monoclonal antibody.

In still yet further aspects of the above improvement, the anti-IgG4 antibody is selected from the group consisting of: 3H293, 0.B.27, 5C7, IGHG4/1345 and IGHG4/2042A.

In still further aspects of the above improvement, the solid support is a microparticle. In some aspects, the microparticle comprises latex.

In yet further aspects of the above improvement, the assay is performed in about 1 minute, in about 5 minutes, in about 10 minutes, in about 15 minutes or in about 20 minutes.

In yet still further aspects of the above improvement, the biological sample is whole blood, serum, or plasma.

In yet still further aspects of the above improvement, the assay is an immunoassay or a clinical chemistry assay.

In yet still further aspects of the above improvement, the method is performed using single molecule detection, lateral flow, or a point-of care method.

In a further embodiment, the present disclosure relates to the use of an anti-IgG4 antibody capture reagent for detecting immunoglobulin G, subclass 4 (IgG4) in a biological sample, wherein the anti-IgG4 antibody capture reagent comprises an anti-IgG4 antibody immobilized on a solid support, wherein the anti-IgG4 antibody does not cross-react with other IgG subclasses.

In some aspects of the above use, the anti-IgG4 antibody is a monospecific antibody.

In other aspects of the above use, the monospecific antibody is directed to a linear epitope. In still further aspects of the above use, the monospecific antibody is directed to a conformational epitope.

In yet still further aspects of the above use, the anti-IgG4 antibody is a monoclonal antibody.

In yet still further aspects of the above use, the anti-IgG4 antibody is selected from the group consisting of: 3H293, 0.B.27, 5C7, IGHG4/1345 and IGHG4/2042A.

In yet still further aspects of the above use, the solid support is a microparticle. In still further aspects of the above use, the microparticle comprises latex.

In yet still further aspects of the above use, the assay is performed in about 1 minute, in about 5 minutes, in about 10 minutes, in about 15 minutes or in about 20 minutes.

In yet still further aspects of the above use, the biological sample is whole blood, serum, or plasma.

Other aspects and aspects of the disclosure will be apparent in light of the following detailed description and accompanying figures.

### BRIEF DESCRIPTION OF THE DRAWINGS

FIGS. 1A-1D are exemplary plots depicting average absorbance of detection of IgG subclass antigens vs. screened mAb concentrations. FIGS. 1A and 1B show IgG4 specific antibodies with minimal to no cross reactivity with IgG1-3. FIGS. 1C and 1D show IgG4 specific antibodies with considerable cross reactivity with IgG1-3.

### DETAILED DESCRIPTION

The present disclosure relates to methods, kits, and systems to detect the presence of or determine the amount, concentration and/or level of immunoglobulin G, subclass 4 (IgG4) in a sample.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other aspects “comprising,” “consisting of” and “consisting essentially of,” the aspects or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Antibody” and “antibodies” as used herein refers to monoclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11): 1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Bead” and “particle” are used herein interchangeably and refer to a substantially spherical solid support. One example of a bead or particle is a microparticle. Microparticles that can be used herein can be any type known in the art.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1,” “CDR2,” and “CDR3,” for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1,” “L2,” and “L3,” or “H1,” “H2,” and “H3,” where the “L” and the “H” designate the light chain and the heavy chain regions, respectively. These regions may be referred to as “Chothia CDRs,” which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain aspects use Kabat- or Chothia-defined CDRs.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international IMMUNOGENETICS® (IMGT®) information system (hypertext transfer protocol://imgt.cines.fr/texts/IMGTrepertoire/LocusGenes/).

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

“Monoclonal antibody” as used herein refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, Ill.) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA).

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low,” “medium,” or “high” levels), can be used. Multiple calibrators (i.e., more than one calibrator or a varying amount of calibrator(s)) can be used in conjunction to comprise a “sensitivity panel.”

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-IG®s, and other antibodies as described herein (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Reference level” as used herein refers to an assay cutoff value (or level) that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). As used herein, the term “cutoff” refers to a limit (e.g., such as a number) above which there is a certain or specific clinical outcome and below which there is a different certain or specific clinical outcome.

This disclosure provides exemplary reference levels. However, it is well-known that reference levels may vary depending on the nature of the immunoassay (e.g., capture and detection reagents employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

“Sample,” “test sample,” “specimen,” “sample from a subject,” “biological sample,” and “patient sample” may be used interchangeably herein to refer to a sample of blood, such as whole blood (including for example, capillary blood, venous blood, dried blood spot, etc.), tissue, urine, serum, plasma, amniotic fluid, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Additionally, the sample can be a nasopharyngeal or oropharyngeal sample obtained using one or more swabs that, once obtained, is placed in a sterile tube containing a virus transport media (VTM) or universal transport media (UTM), for testing. Further, the sample can be a nasal mucus specimen.

A variety of cell types, tissue, or bodily fluid may be utilized to obtain a sample. Such cell types, tissues, and fluid may include sections of tissues such as biopsy and autopsy samples, nasal mucus specimens, oropharyngeal specimens, nasopharyngeal specimens, frozen sections taken for histologic purposes, blood (such as whole blood, dried blood spots, etc.), plasma, serum, red blood cells, platelets, interstitial fluid, cerebrospinal fluid, etc. Cell types and tissues may also include lymph fluid, cerebrospinal fluid, or any fluid collected by aspiration. A tissue or cell type may be provided by removing a sample of cells from a human and a non-human animal, but also can be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some aspects, the sample is a whole blood sample. In some aspects, the sample is a capillary blood sample. In some aspects, the sample is a dried blood spot. In some aspects, the sample is a serum sample. In yet other aspects, the sample is a plasma sample. In some aspects, the sample is an oropharyngeal specimen. In other aspects, the sample is a nasopharyngeal specimen. In other aspects, the sample is sputum. In other aspects, the sample is endotracheal aspirate. In still yet other aspects, the sample is bronchoalveolar lavage. In yet other aspects, the sample is a nasal mucus specimen.

As used herein the term “single molecule detection” refers to the detection and/or measurement of a single molecule of an analyte in a test sample at very low levels of concentration (such as pg/mL or femtogram/mL levels). A number of different single molecule analyzers or devices are known in the art and include nanopore and nanowell devices. Examples of nanopore devices are described in PCT International Application WO 2016/161402, which is hereby incorporated by reference in its entirety. Examples of nanowell device are described in PCT International Application WO 2016/161400, which is hereby incorporated by reference in its entirety.

“Solid phase” or “solid support” as used interchangeably herein, refers to any material that can be used to attach and/or attract and immobilize an antibody capture reagent, or a binding partner. The solid phase can be chosen for its intrinsic ability to attract and immobilize a capture agent. Alternatively, the solid phase can have affixed thereto a linking agent that has the ability to attract and immobilize the capture agent or capture specific binding partner, or a binding partner. For example, the linking agent can include a charged substance that is oppositely charged with respect to the capture agent or a specific binding partner itself or to a charged substance conjugated to the capture agent or specific binding partner. In general, the linking agent can be any binding partner (preferably specific) that is immobilized on (attached to) the solid phase and that has the ability to immobilize the capture agent or specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

“Specific binding partner” or “Specific binding member,” as used interchangeable herein, is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human). In some aspects, the subject may be a human, a non-human primate, or a cat. In some aspects, the subject is a human. The subject or patient may be undergoing other forms of treatment. In some aspects, the subject is a human that may be undergoing other forms of treatment.

As used herein, a “system” refers to a plurality of real and/or abstract elements operating together for a common purpose. In some aspects, a “system” is an integrated assemblage of hardware and/or software elements. In some aspects, each component of the system interacts with one or more other elements and/or is related to one or more other elements. In some aspects, a system refers to a combination of components and software for controlling and directing methods.

As used herein, the term “test strip” can include one or more bibulous or non-bibulous materials. If a test strip comprises more than one material, the one or more materials are preferably in fluid communication. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, dacron, PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH, salt concentration, or specific gravity of an applied sample to optimize test conditions.

The material or materials can be a single structure such as a sheet cut into strips or it can be several strips or particulate material bound to a support or solid surface such as found, for example, in thin-layer chromatography and may have an absorbent pad cither as an integral part or in liquid contact. The material also can be a sheet having lanes thereon, capable of spotting to induce lane formation, wherein a separate assay can be conducted in each lane. The material can have a rectangular, circular, oval, triangular, or other shape provided that there is at least one direction of traversal of a test solution by capillary migration. Other directions of traversal may occur such as in an oval or circular piece contacted in the center with the test solution. However, the main consideration is that there be at least one direction of flow to a predetermined site.

The support for the test strip, where a support is desired or necessary, will normally be water insoluble, frequently non-porous and rigid but may be clastic, usually hydrophobic, and porous and usually will be of the same length and width as the strip but may be larger or smaller. The support material can be transparent, and, when a test device of the present technology is assembled, a transparent support material can be on the side of the test strip that can be viewed by the user, such that the transparent support material forms a protective layer over the test strip where it may be exposed to the external environment, such as by an aperture in the front of a test device. A wide variety of non-mobilizable and non-mobilizable materials, both natural and synthetic, and combinations thereof, may be employed provided only that the support does not interfere with the capillary action of the material or materials, or non-specifically bind assay components, or interfere with the signal producing system. Illustrative polymers include polyethylene, polypropylene, poly(4-methylbutene), polystyrene, polymethacrylate, poly(ethylene terephthalate), nylon, poly(vinyl butyrate), glass, ceramics, metals, and the like. Elastic supports may be made of polyurethane, neoprene, latex, silicone rubber and the like.

### 2. Methods for Detecting Immunoglobulin G, Subclass 5 (IgG4)

The present disclosure relates to methods of detecting an immunoglobulin G, subclass 4 (IgG4) in a biological sample. The method comprises adding an anti-IgG4 antibody capture reagent to the biological sample. The present disclosure further relates to an improvement in the method in that the method comprises using an anti-IgG4 antibody that does not cross-react with other IgG subclasses.

Examples of antibodies that can be used in the methods described herein include the antibodies provided in the below Table A:

TABLE A

| Vendor | Catalog No./Product Code | Clone | Antibody |
|---|---|---|---|
| enQuire BioReagents | 3503-RBM2-P1ABX | IGHG4/2042A | Rabbit IgG |
| (Littleton, CO) | | | |
| enQuire BioReagents | 3503-MSM1-P2 | IGHG4/1345 | Mouse IgG1 |
| (Littelton, CO) | | | |
| Abcam | ab1930 | 5C7 | Mouse IgG1 |
| (Cambridge, UK) | | | |
| US Biological Life | I1904-89A | 0.B.27 | Mouse IgG1 |
| Sciences | | | |
| (Swampscott, MA) | | | |
| US Biological Life | I1904-89B | 3H293 | Mouse IgG1(κ) |
| Sciences | | | |
| (Swampscott, MA) | | | |

In some embodiments, the anti-IgG4 antibody capture reagent comprises an anti-IgG4 antibody immobilized on a solid support. The anti-IgG4 antibody may be immobilized onto a variety of supports, such as magnetic or chromatographic matrix particles, the surface of an assay plate (such as microtiter wells), pieces of a solid substrate material, particles or beads, and the like. In some embodiments, the anti-IgG4 antibody capture reagent comprises a microparticle. In some embodiments, the particle comprises latex.

The nature of methods described herein is not critical and can be conducted using any assay known in the art such as, for example, immunoassays, lateral flow assays, protein immunoprecipitation, turbidimetric, immunoelectrophoresis, chemical analysis, SDS-PAGE and Western blot analysis, protein immunostaining, electrophoresis analysis, a protein assay, a competitive binding assay, or a functional protein assay. Also, the assay can be employed in a clinical chemistry format such as would be known by one of ordinary skill in the art and described herein. It is known in the art that the values (e.g., reference levels, cutoffs, thresholds, specificities, sensitivities, concentrations of calibrators and/or controls etc.) used in an assay that employs a specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4th edition, chapter 3.5, pages 315-322, the contents of which are herein incorporated by reference).

Other methods of detection include the use of, or can be adapted for use on, a nanopore device or nanowell device, e.g., for single molecule detection. Examples of nanopore devices are described in International Patent Publication No. WO 2016/161402, which is hereby incorporated by reference in its entirety. Examples of nanowell devices are described in International Patent Publication No. WO 2016/161400, which is hereby incorporated by reference in its entirety. Other devices and methods appropriate for single molecule detection also can be employed.

In some embodiments, the biological sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

### Preparation/Production Methods for Antibodies for Use as a Capture Reagent

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including DHFR-CHO cells, described in Urlaub and Chasin, Proc. Natl. Acad. Sci. USA, 77: 4216-4220 (1980)), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In some embodiments, the purification of the antibodies can be done in CHO and/or HEK cells using routine techniques known in the art.

Host cells also can be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies.

In a preferred system for recombinant expression of an antibody, or antigen-binding portion thereof, a recombinant expression vector encoding both the antibody heavy chain and the antibody light chain is introduced into DHFR-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH:VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass); Gray et al. (1995) J. Imm. Meth. 182:155-163; Kenny et al. (1995) Bio/Technol. 13:787-790); and B-cell selection (Steenbakkers et al. (1994) Molec. Biol. Reports 19:125-134 (1994)).

An affinity matured antibody may be produced by any one of a number of procedures that are known in the art. For example, Marks et al., BioTechnology, 10: 779-783 (1992) describes affinity maturation by VH and VL domain shuffling. Random mutagenesis of CDR and/or framework residues is described in Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); Hawkins et al, J. Mol. Biol., 226: 889-896 (1992). Selective mutation at selective mutagenesis positions and at contact or hypermutation positions with an activity enhancing amino acid residue is described in U.S. Pat. No. 6,914,128 B1.

Antibody fragments or variants thereof also can be prepared by delivering a polynucleotide encoding an antibody to a suitable host, so as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody fragments or variants thereof also can be prepared by delivering a polynucleotide to provide transgenic plants and cultured plant cells (e.g., tobacco, maize, and duckweed) that produce such antibodies, specified portions or variants in the plant parts or in cells cultured therefrom. For example, Cramer et al. (1999) Curr. Top. Microbiol. Immunol. 240:95-118, and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. Sec, e.g., Hood et al., Adv. Exp. Med. Biol. (1999) 464:127-147 and references cited therein. Antibody fragments or variants thereof have also been produced in large amounts from transgenic plant seeds including antibody fragments, such as single chain antibodies (scFv's), using, for example, tobacco seeds and potato tubers. See, e.g., Conrad et al. (1998) Plant Mol. Biol. 38:101-109 and references cited therein. Thus, antibodies also can be produced using transgenic plants according to known methods.

Antibody derivatives can be produced, for example, by adding exogenous sequences to modify immunogenicity or to reduce, enhance, or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein such fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al., which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8(10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxyapatite chromatography, and lectin chromatography. High performance liquid chromatography (“HPLC”) also can be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

### Variations on Methods

The disclosed methods detect the presence or determine the amount or level of immunoglobulin G, subclass 4 (IgG4) present in a biological sample as described herein. The methods may also be adapted in view of other methods for analyzing analytes. Examples of well-known variations include, but are not limited to, immunoassay, competitive inhibition immunoassay (e.g., forward and reverse), enzyme multiplied immunoassay technique (EMIT), a competitive binding assay, bioluminescence resonance energy transfer (BRET), one-step antibody detection assay, homogenous assay, heterogeneous assay, capture on the fly assay, single molecule detection assay, lateral flow assay, turbidimetry (immunoturbidimetry), nephelometry (immunonephelometry), etc.

Immunoassay. The analyte of interest, such as the immunoglobulin G, subclass 4 (IgG4), as described above, may be analyzed using the anti-IgG4 capture reagent, as described above, in an immunoassay. The presence or amount of the immunoglobulin G, subclass 4 present in a biological sample may be readily determined using an immunoassay. For example, in one aspect, one method that can be used is a chemiluminescent microparticle immunoassay, in particular one employing the ARCHITECT® automated analyzer (Abbott Laboratories, Abbott Park, Ill.). Other methods that can be used include, for example, mass spectrometry, and immunohistochemistry (e.g., with sections from tissue biopsies). Additionally, methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of an antibody to the immunoglobulin G, subclass 4 can be detected via direct labels, such as fluorescent or luminescent tags, metals and radionuclides attached to the antibody or via indirect labels, such as alkaline phosphatase or horseradish peroxidase.

In some embodiments, the anti-IgG4 antibody capture reagent is immobilized on a solid support. The anti-IgG4 antibody capture reagent may be immobilized onto a variety of supports, such as magnetic or chromatographic matrix particles, the surface of an assay plate (such as microtiter wells), pieces of a solid substrate material, and the like. An assay strip can be prepared by coating the antigen and/or antibody or plurality of antibodies in an array on a solid support. This strip can then be dipped into the test sample and processed quickly through washes and detection steps to generate a measurable signal, such as a colored spot.

One-Step Immunoassay or “Capture on the Fly” Assay. In a capture on the fly immunoassay, a solid substrate is pre-coated with an immobilization agent. The capture composition, the analyte and the detection composition are added to the solid substrate together, followed by a wash step prior to detection. The capture composition can bind the analyte and comprises a ligand or property that interacts with the immobilization agent. The capture composition and the detection composition may comprise any moiety capable of capture or detection as described herein or known in the art.

The solid support and the capture and detection compositions may be added to a test sample (either sequentially or simultaneously). The ligand on the at least two different types of microparticle reagents binds to the immobilization agent on the solid support to form a solid support/microparticle reagent complex. Any analyte of interest present in the sample binds to the solid support/microparticle reagent complex to form a solid support/microparticle reagent/analyte complex. The detection reagent binds to the solid support/microparticle reagent/analyte complex and the detectable label is detected. An optional wash step may be employed before the detection. In certain embodiments, in a one-step assay more than one analyte may be measured.

The use of a capture on the fly assay can be done in a variety of formats as described herein, and known in the art. For example, the format can be a sandwich assay, but alternately can be a competition assay, can employ any number of microparticle reagents, or use other variations such as are known.

Forward Competitive Inhibition Assay. In a forward competitive format, an aliquot of labeled immunoglobulin G, subclass 4 (IgG4) having a fluorescent label, a tag attached with a cleavable linker, etc.) of a known concentration is used to compete with immunoglobulin G, subclass 4 (IgG4) from a biological sample for binding to antibody directed against IgG4.

In a forward competition assay, a microparticle reagent can either be sequentially or simultaneously contacted with the biological sample and a labeled IgG4, or fragment thereof. The IgG4, or fragment thereof, can be labeled with any detectable label, including a detectable label comprised of tag attached with a cleavable linker.

The labeled IgG4, or fragment thereof, the biological sample and the microparticle reagent may be incubated at a pH of from about 4.5 to about 10.0, at a temperature of from about 2ºC to about 45° C., and for a period from at least one (1) minute to about eighteen (18) hours, from about 2-6 minutes, from about 7-12 minutes, from about 5-15 minutes, or from about 3-4 minutes. The amount of detectable label complexed with the antibody directed against IgG4 is then quantified., thereby determining the concentration of IgG4 in the biological sample.

Reverse Competitive Inhibition Assay. Similar to the forward competitive format, a reverse competition assay, an immobilized analyte of interest can either be sequentially or simultaneously contacted with a test sample and at least one labeled specific binding partner. The analyte of interest can be bound to a solid support, such as the solid supports discussed above.

The immobilized analyte of interest, biological sample and at least one labeled specific binding partner are incubated under conditions similar to those described above. Two different types of complexes are then generated. Specifically, one of the analyte of interest-specific binding partner complexes generated is immobilized and contains a detectable label (e.g., a fluorescent label, etc.) while the other analyte of interest-specific binding partner complex is not immobilized and contains a detectable label. The non-immobilized analyte of interest-specific binding partner complex and the remainder of the biological sample are removed from the presence of the immobilized analyte of interest-specific binding partner complex through techniques known in the art, such as washing. Once the non-immobilized analyte of interest-specific binding partner complex is removed, the amount of detectable label in the immobilized analyte of interest-specific binding partner complex is then quantified. The concentration of analyte of interest in the test sample can then be determined by comparing the quantity of detectable label.

Single Molecule Detection Assay. Single molecule detection assays and methods, such as the use of a nanopore device or nanowell device, also can be used. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety. Other devices and methods appropriate for single molecule detection also can be employed.

Lateral Flow Assays. Lateral flow assays are generally provided in a device comprising a lateral flow test strip (e.g., nitrocellulose or filter paper), a sample application area (e.g., sample pad), a test results area (e.g., a test line), an optional control results area (e.g., a control line), and an analyte-specific binding partner (e.g., the anti-IgG4 antibody capture reagent) that is bound to a detectable label (e.g., a colored particle or an enzyme detection system). Sec, e.g., U.S. Pat. Nos. 6,485,982; 6,187,598; 5,622,871; 6,565,808; and 6,809,687; and U.S. patent application Ser. No. 10/717,082, each of which is incorporated herein by reference.

In some embodiments, the present disclosure provides assays for detecting IgG4 in a sample. In some embodiments, the technology relates to analytical devices that are suitable for use in the home, clinic, or hospital, and that are intended to give an analytical result that is rapid with minimum degree of skill and involvement from the user. In some embodiments, use of the devices described herein involves methods in which a user performs a sequence of operations to provide an observable test result.

In some embodiments, also provided is a test device comprising a reagent-impregnated test strip to provide a specific binding assay, e.g., an immunoassay. In some embodiments, a sample is applied to one portion of the test strip and is allowed to permeate through the strip material, usually with the aid of an eluting solvent such as water and/or a suitable buffer (e.g., an extraction buffer optionally comprising a detergent). In so doing, the sample progresses into or through a detection zone in the test strip wherein the analyte suspected of being in the sample (e.g., IgG4) is immobilized. Analyte present in the sample can therefore become bound within the detection zone. The extent to which the analyte becomes bound in that zone can be determined with the aid of labelled reagents that also can be incorporated in the test strip or applied thereto subsequently.

In some embodiments, the analytical test device comprises a hollow casing constructed of moisture-impervious solid material containing a dry porous carrier that communicates directly or indirectly with the exterior of the casing such that a liquid test sample can be applied to the porous carrier. Another aspect relates to a device that comprises a porous solid phase material carrying in a first zone the detection reagent that is retained in the first zone while the porous material is in the dry state but is free to migrate through the porous material when the porous material is moistened, for example, by the application of an aqueous liquid sample suspected of containing the analyte. In some embodiments, the porous material comprises in a second zone, which is spatially distinct from the first zone, comprising the microparticle reagents having specificity for the analyte and which is capable of participating with the detection reagent in either a “sandwich” or a “competition” reaction. The microparticle reagents is firmly immobilized on the porous material such that it is not free to migrate when the porous material is in the moist state. In some embodiments, a device as described herein is contacted with an aqueous liquid sample suspected of containing the analyte, such that the sample permeates by capillary action through the porous solid phase material via the first zone into the second zone and the labelled reagent migrates therewith from the first zone to the second zone, the presence of analyte in the sample being determined.

Examples of lateral flow assays that can be used in the present disclosure include, for example, PANBIOR, BINAX® and BINAXNOW® (Alere, Abbott Park, Ill.).

Turbidimetric Assays. Turbidimetry (immunoturbidimetry) and nephelometry (immunonephelometry) measurements are commonly used to quantify immune-complex precipitates in solution by their ability to interact with incident light. Nephelometric or turbidimetric immunoassay systems operate where the concentration of antibody (e.g., anti-IgG4 antibody) is held constant and the amount of complex formed depends directly on the concentration of antigen (e.g., IgG4) in the mixture. This permits the formation of complexes of a constant size, providing a reproducible, stoichiometric relationship between the number of complexes formed at a given antigen (e.g., IgG4) concentration. Polymers or hydrophilic agents may be added to accelerate the immunoprecipitation in both nephelometry and turbidimetry

For turbidimetric measurements, the detector is oriented at a straight-on 180-degree angle relative to the incident light source. With this experimental design, the decrease in light intensity is quantified and the amount of light passing through the solution to the detector is inversely proportional to the concentration of antigen (e.g., IgG4) in the sample. Quantification is accomplished by comparing the signal from the sample with that of a standard curve. Nephelometry detects immune-complex precipitates by their ability to reflect light, a phenomenon termed “Rayleigh light scattering.” Because nephelometry measures light scatter, the light detector is oriented at an angle (e.g., 30 or 90 degrees) relative to the incident light source. The amount of light reaching the detector in nephelometry is directly proportional to the quantity of antigen (e.g., IgG4) in the sample. Quantification is accomplished by comparing the signal from samples with a standard curve.

### Samples and Controls

Test or Biological Sample. As used herein, the terms “sample,” “test sample,” and “biological sample” refer to a fluid sample containing immunoglobulin G, subclass 4 (IgG4). The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing immunoglobulin G, subclass 4 (IgG4) may be assayed directly. In a particular example, the source of immunoglobulin G, subclass 4 (IgG4) is a mammalian (e.g., human) bodily substance (e.g., a tissue) or bodily fluid. Examples of suitable bodily substances or fluids include, but are not limited to, whole blood (including, for example, capillary blood, venous blood, etc.), serum, plasma, urine, saliva, sweat, sputum, semen, mucus, lacrimal fluid, lymph fluid, amniotic fluid, interstitial fluid, nasal mucus specimens, lower respiratory specimens (e.g., endotracheal aspirate or bronchoalveolar lavage), cerebrospinal fluid, feces, one or more dried blood spots, etc. Tissues may include, but are not limited to, oropharyngeal specimens, nasopharyngeal specimens, skeletal muscle tissue, liver tissue, lung tissue, kidney tissue, myocardial tissue, brain tissue, bone marrow, cervix tissue, skin, etc. The sample may be a liquid extract of a solid sample. In certain cases, the source of the sample may be an organ or tissue, such as a biopsy sample, which may be solubilized by tissue disintegration/cell lysis.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

As discussed above, a fluid sample may be diluted prior to use in an assay. For example, in embodiments where the source of the immunoglobulin G, subclass 4 (IgG4) is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, a fluid sample is not diluted prior to use in an assay. In some embodiments, the diluent may optionally contain an antibody, such as a non-IgG4 subclass antibody to remove at least a portion of the other subclass of IgG not of interest in the assay.

In some cases, the sample may undergo pre-analytical processing or pre-treatment. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art.

In some cases, the fluid sample may be concentrated prior to use in an assay. For example, in embodiments where the source of immunoglobulin G, subclass 4 (IgG4) is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

Controls and Calibrators. It may be desirable to include a control (such as a positive and/or negative control, which are well known in the art). For example, a positive control can be in vivo purified or recombinant IgG4, or a fragment thereof that binds to the anti-IgG4 antibody capture reagent. In some embodiments, the positive control can be a full-length IgG4. Alternatively, the positive control can be a fragment or variant of the full-length IgG4. In some embodiments, a control or calibrator can be IgG4 that is produced recombinantly, using methods known in the art.

The control may be analyzed separately from, or concurrently with, the sample from the subject as described above. The results obtained from the subject sample can be compared to the results or information obtained from the control sample. Standard curves may be provided or developed with use of the calibrators and controls, with which assay results for the sample may be compared. Such standard curves typically present levels of marker as a function of assay units (i.e., fluorescent signal intensity, if a fluorescent label is used).

It may also be desirable to include one or more calibrators for use in calibrating of any automated or semi-automated system for which the methods and kits described herein are adapted for use. The use of calibrators in such systems is well known in the art. For example, one or more calibrators can include IgG4, or a fragment thereof.

The calibrator may optionally be part of a series of calibrators in which each of the calibrators differs from the other calibrators in the series by concentration of the IgG4 or fragment thereof.

The time required for the assay is dependent on any of all of the type of assay, the type of sample, the desired levels of stringency, accuracy, and specificity. Preferably, the assay is performed in less than one hour. In some aspects, the assay is performed in about 1 minute. In some aspects, the assay is performed in about 5 minutes. In some aspects, the assay is performed in about 10 minutes. In some aspects, the assay is performed in about 15 minutes, In some aspects, the assay is performed in about 20 minutes. Thus, in certain embodiments, the assay is performed in 1 to 20 minutes, 5 to 20 minutes, 10 to 20 minutes, 15 to 20 minutes, 1 to 15 minutes, 5 to 15 minutes, 5 to 10 minutes, 1 to 10 minutes, 5 to 10 minutes, or 1 to 5 minutes.

### Kit

Provided herein is a kit, which may be used in the methods described herein for assaying or assessing a test sample for immunoglobulin G, subclass 4 (IgG4). The kit comprises at least one component for assaying the test sample for IgG4, as well as instructions for assaying the test sample for IgG4. For example, the kit can comprise instructions for assaying the test sample for IgG4 using an immunoassay. Instructions included in kits can be affixed to packaging material, can be included as a package insert, or can be viewed or downloaded from a particular website that is recited as part of the kit packaging or inserted materials. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component for assaying the test sample for immunoglobulin G, subclass 4 (IgG4) may include an anti-IgG4 antibody or a solid support or a combination thereof. The kit further can include purified, recombinant, and/or labeled IgG4, for use as calibrators or controls, or optionally, these can be provided separately.

Alternatively, or additionally, the kit can comprise a calibrator or control, as described previously herein, and/or at least one container (e.g., tubes, microtiter plates or strips) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay.

The kit may further comprise reference standards for quantifying IgG4. The reference standards may be employed to establish standard curves for interpolation and/or extrapolation of the IgG4 concentration. The instructions also can include instructions for generating a standard curve.

Any IgG4 antibodies or anti-IgG4 antibodies, which are provided in the kit, can incorporate a detectable label, such as a fluorophore, radioactive moiety, enzyme, biotin/avidin label, chromophore, chemiluminescent label, or the like, or the kit can include reagents for labeling the components of the kit.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit also can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents or extraction buffers), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., tubes and microtiter plates. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit also can include one or more instrument for assisting with obtaining a test sample, such as a syringe, pipette, forceps, measured spoon, or the like.

The kit also can include one or more sample collection/acquisition instruments for assisting with obtaining a test sample (e.g., microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, surgical knife or laser (e.g., particularly hand-held), syringes, sterile container, or canula, for obtaining, storing, or aspirating tissue samples).

If desired, the kit can further comprise one or more components, alone or in further combination with instructions, for assaying the test sample for another analyte, including for example biomarkers or markers for other infectious agents.

### Adaptation of Kit and Method

The kit (or components thereof), as well as the method for detecting the presence or determining the amount or level or concentration of immunoglobulin G, subclass 4 (IgG4) in a test sample by an immunoassay as described herein, can be adapted for use in a variety of automated and semi-automated systems or platforms (including those wherein the solid phase comprises a microparticle), as described in, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, Ill.) as Abbott Point of Care (i-STAT® or i-STAT Alinity, Abbott Laboratories) as well as those described in U.S. Pat. Nos. 5,089,424 and 5,006,309, and as commercially marketed, e.g., by Abbott Laboratories (Abbott Park, Ill.) as ARCHITECT® or the series of Abbott Alinity devices. Such systems include one or more devices and/or components that can be used to detect one or more labels in the resulting complexes formed in the methods described previously herein.

Some of the differences between an automated or semi-automated system as compared to a non-automated system include the substrate to which the first specific binding partner (e.g., recombinant antigen or capture reagent) is attached, and the length and timing of the capture, detection, and/or any optional wash steps. Whereas a non-automated format may require a relatively longer incubation time with test sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories that may be used include, but are not limited to, AXSYM®, IMX® (see, e.g., U.S. Pat. No. 5,294,404, which is hereby incorporated by reference in its entirety), PRISM®, EIA (bead), and QUANTUM™ II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described in, for example, U.S. Pat. No. 5,063,081, U.S. Patent App. Publication Nos. 2003/0170881, 2004/0018577, 2005/0054078, and 2006/0160164, which are incorporated by reference herein in their entireties.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048, improved signal generation may be obtained, e.g., in an i-STAT® cartridge format, using a nucleic acid sequence linked to the signal antibody as a signal amplifier.

### Analysis and Interpretation of Results

The results obtained using the methods of the present disclosure can be analyzed and interpreted individually or in combination with other any other results obtained prior to, during or after the results of the methods of the present disclosure are performed. The nature of the other results analyzed and interpreted with the results of the present disclosure are changeable.

It will be readily apparent to those skilled in the art that other suitable modifications and adaptations of the methods of the present disclosure described herein are readily applicable and appreciable, and may be made using suitable equivalents without departing from the scope of the present disclosure or the embodiments and embodiments disclosed herein. The disclosures of all journal references, U.S. patents, and publications referred to herein are hereby incorporated by reference in their entireties.

### Example 1

Anti-IgG4 mAbs (Table 1) were screened using a sandwich ELISA. The target antibodies were coated on the base of a microwell plate at 2 μg/mL in PBS. Serially-diluted samples and controls were diluted in a blocking buffer and incubated with the antibodies. Horse radish peroxidase labeled species specific conjugates were used for detection and developed using standard methods.

TABLE 1
Monoclonal Human IgG4 Antibodies

| # | Vendor | Catalog No./Product No. | Clone | Antibody |
|---|---|---|---|---|
| 1 | enQuire BioReagents | 3503-RBM2-P1ABX | IGHG4/2042A | Rabbit IgG |
| | (Littleton, CO) | | | |
| 2 | enQuire BioReagents | 3503-MSM1-P2 | IGHG4/1345 | Mouse IgG1 |
| | (Littleton, CO) | | | |
| 3 | Invitrogen | SA5-10205 | RM120 | Rabbit IgG |
| | (Waltham, MA) | | | |
| 4 | Invitrogen | SA5-10206 | RM217 | Rabbit IgG |
| | (Waltham, MA) | | | |
| 5 | LifeSpan Bio | LS-C489989 | IHCG4-1 | Mouse IgG1 |
| | (Seattle, WA) | | | |
| 6 | Abcam, | ab1930 | 5C7 | Mouse IgG1 |
| | (Cambridge, UK) | | | |
| 7 | R&D Systems | MAB98951 | 2350A | Rabbit IgG |
| | (Minneapolis, MN) | | | |
| 8 | R&D Systems | MAB98985 | 985547 | Mouse IgG2a |
| | (Minneapolis, MN) | | | |
| 9 | US Biological Life | I1904-89-HRP | 8.F.178 | Mouse IgG1 |
| | Sciences | | | |
| | (Swampscott, MA) | | | |
| 10 | US Biological Life | I1904-89A | 0.B.27 | Mouse IgG1 |
| | Sciences | | | |
| | (Swampscott, MA) | | | |
| 11 | US Biological Life | I1904-89B | 3H293 | Mouse IgG1(κ) |
| | Sciences | | | |
| | (Swampscott, MA) | | | |

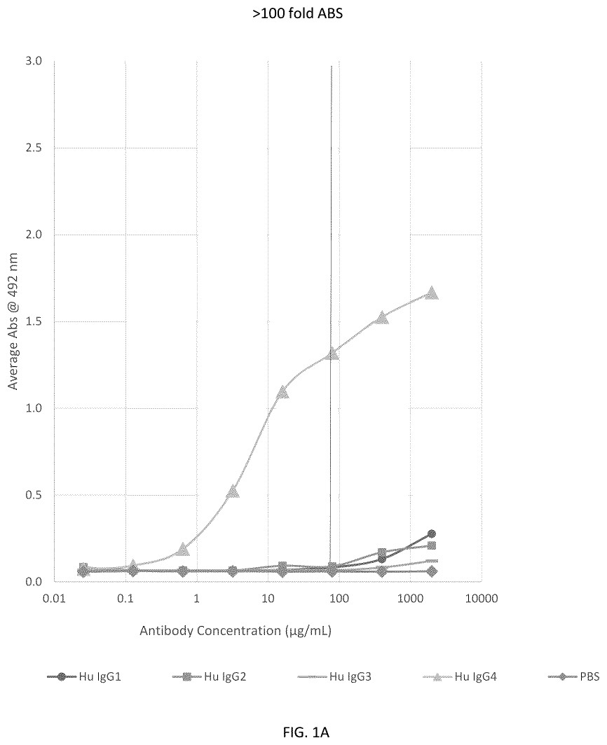

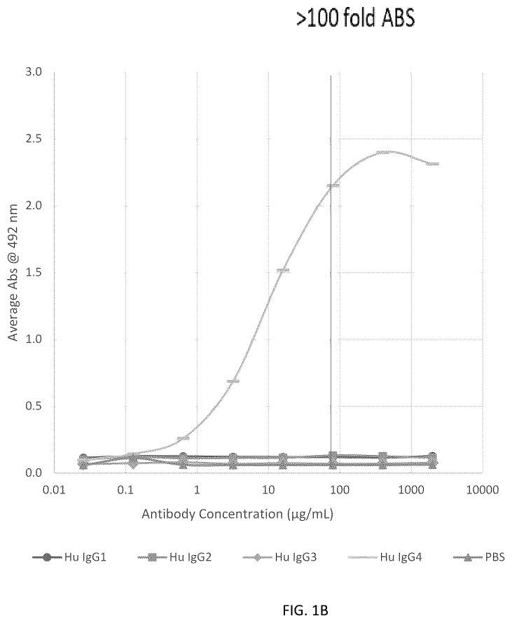

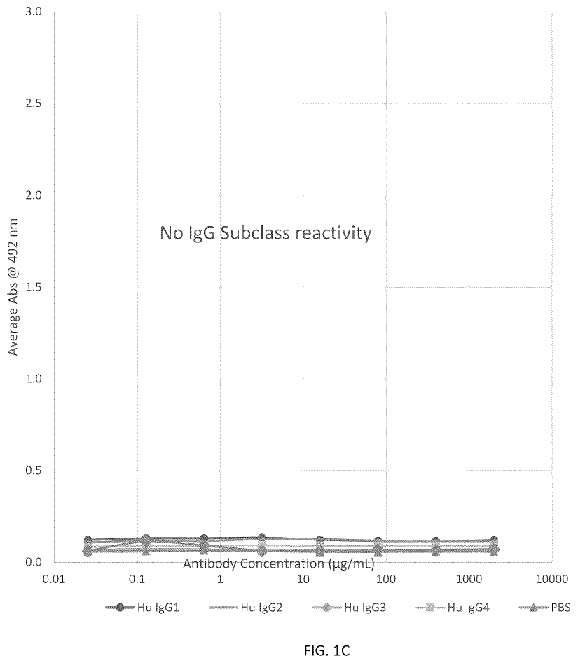

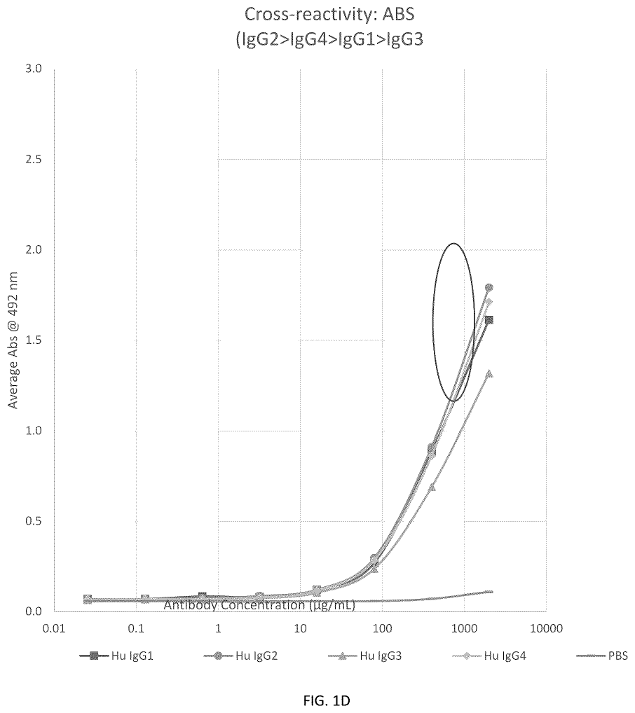

Five of the eleven antibodies shown in Table 1 above had a greater than 100-fold different in average absorbance (ABS) for IgG4 detection over IgG1-3 detection at any given concentration. These were antibody numbers 1-2, 6, 10 and 11, namely, the antibody clones IGHG4/2042A and IGHG4/1245 from enquire BioReagents, antibody clone 5C7 from LifeSpan (LS) Bio, and antibody clones 0.B.27 and 3H293 from US Biological Life Sciences. The exemplary antibodies are shown in FIGS. 1A and 1B. The remaining six antibodies (namely, antibody numbers 3-5 and 7-9 in the above Table) did not achieve a greater than 100-fold different in average absorbance (ABS) for IgG4 detection over IgG1-3 detection (FIGS. 1C and 1D).

The five antibodies (e.g., antibody numbers 1-2, 6 and 10-11 in above Table 1) were coated on <1 μm sized latex microparticles using EDAC coupling chemistry and tested in latex microparticle immunoturbidimetric assays for use on ARCHITECT c8000 clinical chemistry analyzer. In this assay, an exemplary antibody demonstrated agreeable performance metrics: <3% CV for sample from 5.4 to 680 mg/L and achieved the CLSI guidance endogenous interference recovery targets at IgG4 level of ˜400 mg/L.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method of detecting immunoglobulin G, subclass 4 (IgG4) in a biological sample, wherein the method comprises performing an assay on the biological sample obtained from a subject, wherein the assay includes adding an anti-IgG4 antibody capture reagent to the biological sample, wherein the anti-IgG4 antibody capture reagent comprises an anti-IgG4 antibody immobilized on a solid support, wherein the anti-IgG4 antibody does not cross-react with other IgG subclasses.

Clause 2. The method of clause 1, wherein the assay further comprises incubating the anti-IgG4 antibody capture reagent with the biological sample for a period of time.

Clause 3. The method of clause 1 or clause 2, wherein the assay further comprises measuring turbidity.

Clause 4. The method of clause 3, wherein the assay further comprises comparing at least one turbidity measurement to a standard and quantifying an amount of the IgG4 in the biological sample.

Clause 5. The method of clause 4, wherein turbidity is measured by loss of intensity or increase in absorbance of transmitted light through the biological sample at a wavelength of about 570 nm.

Clause 6. The method of any of clauses 1-5, wherein the anti-IgG4 antibody is a monospecific antibody.

Clause 7. The method of clause 6, wherein the monospecific antibody is directed to a linear epitope.

Clause 8. The method of clause 6, wherein the monospecific antibody is directed to a conformational epitope.

Clause 9. The method of any of clauses 1-8, wherein the anti-IgG4 antibody is a monoclonal antibody.

Clause 10. The method of any of clauses 1-9, wherein the anti-IgG4 antibody is selected from the group consisting of: 3H293, 0.B.27, 5C7, IGHG4/1345 and IGHG4/2042A.

Clause 11. The method of any of clauses 1-10, wherein the solid support is a microparticle.

Clause 12. The method of any of clauses 1-11, wherein the microparticle comprises latex.

Clause 13. The method of any of clauses 1-12, wherein the assay is performed in about 1 minute, in about 5 minutes, in about 10 minutes, in about 15 minutes or in about 20 minutes.

Clause 14. The method of any of clauses 1-13, wherein the biological sample is whole blood, serum, or plasma.

Clause 15. The method of any of clauses 1-14, wherein the assay is an immunoassay or a clinical chemistry assay.

Clause 16. The method of any of clauses 1-15, wherein the method is performed using single molecule detection, lateral flow, or a point-of care method.

Clause 17. In an improvement of a method of detecting immunoglobulin G, subclass 4 (IgG4) in a biological sample, wherein the method comprises performing an assay on the biological sample obtained from a subject, wherein the assay includes adding an anti-IgG4 antibody capture reagent to the biological sample, wherein the anti-IgG4 antibody capture reagent comprises an anti-IgG4 antibody immobilized on a solid support, wherein the improvement comprises using an anti-IgG4 antibody that does not cross-react with other IgG subclasses.

Clause 18. The improvement of clause 1, wherein the assay further comprises incubating the anti-IgG4 antibody capture reagent with the biological sample for a period of time.

Clause 19. The improvement of clause 1 or clause 2, wherein the assay further comprises measuring turbidity.

Clause 20. The improvement of clause 3, wherein the method further comprises comparing at least one turbidity measurement to a standard and quantifying an amount of the IgG4 in the biological sample.

Clause 21. The improvement of clause 4, wherein turbidity is measured by loss of intensity or increase in absorbance of transmitted light through the biological sample at a wavelength of about 570 nm.

Clause 22. The improvement of any of clauses 17-21, wherein the anti-IgG4 antibody is a monospecific antibody.

Clause 23. The improvement of clause 22, wherein the monospecific antibody is directed to a linear epitope.

Clause 24. The improvement of clause 22, wherein the monospecific antibody is directed to a conformational epitope.

Clause 25. The improvement of any of clauses 17-24, wherein the anti-IgG4 antibody is a monoclonal antibody.

Clause 26. The improvement of any of claims 17-25, wherein the anti-IgG4 antibody is selected from the group consisting of: 3H293, 0.B.27, 5C7, IGHG4/1345 and IGHG4/2042A.

Clause 27. The improvement of any of clauses 17-26, wherein the solid support is a microparticle.

Clause 28. The improvement of any of clauses 17-27, wherein the microparticle comprises latex.

Clause 29. The improvement of any of clauses 17-28, wherein the assay is performed in about 1 minute, in about 5 minutes, in about 10 minutes, in about 15 minutes or in about 20 minutes.

Clause 30. The improvement of any of clauses 17-29, wherein the biological sample is whole blood, serum, or plasma.

Clause 31. The improvement of any of clauses 17-30, wherein the assay is an immunoassay or a clinical chemistry assay.

Clause 32. The improvement of any of clauses 17-31, wherein the method is performed using single molecule detection, lateral flow, or a point-of care method.

Clause 33. Use of an anti-IgG4 antibody capture reagent for detecting immunoglobulin G, subclass 4 (IgG4) in a biological sample, wherein the anti-IgG4 antibody capture reagent comprises an anti-IgG4 antibody immobilized on a solid support, wherein the anti-IgG4 antibody does not cross-react with other IgG subclasses.

Clause 34. Use of clause 33, wherein the anti-IgG4 antibody is a monospecific antibody.

Clause 35. Use of clause 34, wherein the monospecific antibody is directed to a linear epitope.

Clause 36. Use of clause 34, wherein the monospecific antibody is directed to a conformational epitope.

Clause 37. Use of any of clauses 33-36, wherein the anti-IgG4 antibody is a monoclonal antibody.

Clause 38. The use of any of clauses 33-37, wherein the anti-IgG4 antibody is selected from the group consisting of: 3H293, 0.B.27, 5C7, IGHG4/1345 and IGHG4/2042A.

Clause 39. The use of any of clauses 33-38, wherein the solid support is a microparticle.

Clause 40. The use of any of clauses 33-39, wherein the microparticle comprises latex.

Clause 41. The use of any of clauses 33-40, wherein the assay is performed in about 1 minute, in about 5 minutes, in about 10 minutes, in about 15 minutes or in about 20 minutes.

Clause 42. The use of any of clauses 33-41, wherein the biological sample is whole blood, serum, or plasma.

What is claimed is:

1. A method of detecting immunoglobulin G, subclass 4 (IgG4) in a biological sample, wherein the method comprises performing an assay on the biological sample obtained from a subject, wherein the assay includes adding an anti-IgG4 antibody capture reagent to the biological sample, wherein the anti-IgG4 antibody capture reagent comprises an anti-IgG4 antibody immobilized on a solid support, wherein the anti-IgG4 antibody does not cross-react with other IgG subclasses.

2. The method of claim 1, wherein the assay further comprises incubating the anti-IgG4 antibody capture reagent with the biological sample for a period of time.

3. The method of claim 1, wherein the assay further comprises measuring turbidity.

4. The method of claim 3, wherein the assay further comprises comparing at least one turbidity measurement to a standard and quantifying an amount of the IgG4 in the biological sample.

5. The method of claim 4, wherein turbidity is measured by loss of intensity or increase in absorbance of transmitted light through the biological sample at a wavelength of about 570 nm.

6. The method of claim 1, wherein the anti-IgG4 antibody is a monospecific antibody.

7. The method of claim 6, wherein the monospecific antibody is directed to a linear epitope.

8. The method of claim 6, wherein the monospecific antibody is directed to a conformational epitope.

9. The method of claim 1, wherein the anti-IgG4 antibody is a monoclonal antibody.

10. The method of claim 1, wherein the anti-IgG4 antibody is selected from the group consisting of: 3H293, 0.B.27, 5C7, IGHG4/1345 and IGHG4/2042A.

11. The method of claim 1, wherein the solid support is a microparticle.

12. The method of claim 1, wherein the microparticle comprises latex.

13. The method of claim 1, wherein the assay is performed in about 1 minute, in about 5 minutes, in about 10 minutes, in about 15 minutes or in about 20 minutes.

14. The method of claim 1, wherein the biological sample is whole blood, serum, or plasma.

15. The method of claim 1, wherein the assay is an immunoassay or a clinical chemistry assay.

16. The method of claim 1, wherein the method is performed using single molecule detection, lateral flow, or a point-of care method.

17. In an improvement of a method of detecting immunoglobulin G, subclass 4 (IgG4) in a biological sample, wherein the method comprises performing an assay on the biological sample obtained from a subject, wherein the assay includes adding an anti-IgG4 antibody capture reagent to the biological sample, wherein the anti-IgG4 antibody capture reagent comprises an anti-IgG4 antibody immobilized on a solid support, wherein the improvement comprises using an anti-IgG4 antibody that does not cross-react with other IgG subclasses.

18. The improvement of claim 17, wherein the assay further comprises incubating the anti-IgG4 antibody capture reagent with the biological sample for a period of time.

19. The improvement of claim 17, wherein the assay further comprises measuring turbidity.

20. The improvement of claim 19, wherein the assay further comprises comparing at least one turbidity measurement to a standard and quantifying an amount of the IgG4 in the biological sample.

21. The improvement of claim 20, wherein turbidity is measured by loss of intensity or increase in absorbance of transmitted light through the biological sample at a wavelength of about 570 nm.

22. The improvement of claim 17, wherein the anti-IgG4 antibody is a monospecific antibody.

23. The improvement of claim 22, wherein the monospecific antibody is directed to a linear epitope.

24. The improvement of claim 22, wherein the monospecific antibody is directed to a conformational epitope.

25. The improvement of claim 17, wherein the anti-IgG4 antibody is a monoclonal antibody.

26. The improvement of claim 17, wherein the anti-IgG4 antibody is selected from the group consisting of: 3H293, 0.B.27, 5C7, IGHG4/1345 and IGHG4/2042A.

27. The improvement of claim 17, wherein the solid support is a microparticle.

28. The improvement of claim 17, wherein the microparticle comprises latex.

29. The improvement of claim 17, wherein the assay is performed in about 1 minute, in about 5 minutes, in about 10 minutes, in about 15 minutes or in about 20 minutes.

30. The improvement of claim 17, wherein the biological sample is whole blood, serum, or plasma.

31. The improvement of claim 17, wherein the assay is an immunoassay or a clinical chemistry assay.

32. The improvement of claim 17, wherein the method is performed using single molecule detection, lateral flow, or a point-of care method.

33. Use of an anti-IgG4 antibody capture reagent for detecting immunoglobulin G, subclass 4 (IgG4) in a biological sample, wherein the anti-IgG4 antibody capture reagent comprises an anti-IgG4 antibody immobilized on a solid support, wherein the anti-IgG4 antibody does not cross-react with other IgG subclasses.

34. Use of claim 33, wherein the anti-IgG4 antibody is a monospecific antibody.

35. Use of claim 34, wherein the monospecific antibody is directed to a linear epitope.

36. Use of claim 34, wherein the monospecific antibody is directed to a conformational epitope.

37. Use of claim 33, wherein the anti-IgG4 antibody is a monoclonal antibody.

38. The use of claim 33, wherein the anti-IgG4 antibody is selected from the group consisting of: 3H293, 0.B.27, 5C7, IGHG4/1345 and IGHG4/2042A.

39. The use of claim 33, wherein the solid support is a microparticle.

40. The use of claim 33, wherein the microparticle comprises latex.

41. The use of claim 33, wherein the assay is performed in about 1 minute, in about 5 minutes, in about 10 minutes, in about 15 minutes or in about 20 minutes.

42. The use of claim 33, wherein the biological sample is whole blood, serum, or plasma.
